(19) United States
(12) Patent Application Publication
(10) Pub. No.: US 2011/0306858 A1
(43) Pub. Date: Dec. 15, 2011

(54) Systems And Methods For Wavelet Transform Using Mean-Adjusted Wavelets

(75) Inventors: James N. Watson, Dunfermline (GB); Dominique Jacquel, Edinburgh (GB); Paul Stanley Addison, Edinburgh (GB)

(73) Assignee: Nellcor Puritan Bennett Ireland, Mervue (IE)

(21) Appl. No.: 12/797,747

(22) Filed: Jun. 10, 2010

(51) Int. Cl. A61B 5/1455; G06F 19/00

(52) U.S. Cl. 600/323; 702/66

(57) ABSTRACT

Methods and systems are disclosed for transforming a signal using a continuous wavelet transform based at least in part on a truncated, mean-adjusted wavelet. A wavelet may be truncated to a finite support to generate a truncated wavelet. The real part of the truncated wavelet may be forced to have a zero mean to generate a truncated, mean-adjusted wavelet. The signal may be transformed using a continuous wavelet transform based at least in part on the truncated mean-adjusted wavelet. Information may be derived about the signal from the transformed signal.

### SUMMARY OF THE DISCLOSURE

The present disclosure relates to signal processing and, more particularly, the present disclosure relates to continuous wavelet transforms using truncated and mean-adjusted wavelets for processing, for example, a photoplethysmograph (PPG) signal.

By its nature, the numerical implementation of a continuous wavelet transform (CWT) is an approximation and therefore exhibits certain rounding and other numerical errors. These artifacts, in many circumstances, are negligibly small and may be ignored during subsequent analysis. However, for a time-domain implementation of a continuous wavelet transform, the truncation of the wavelet function to fit a finite support may introduce a significant numerical error.

The problem may be caused by a non-zero mean that manifests in the real or imaginary part of the truncated wavelet function, which, although small, may lead to large errors when convolved with signals and signal segments with significant non-zero means. The present disclosure describes a solution for the numerical error, which may be based at least in part by forcing the real or imaginary part of the complex wavelet to have a zero mean.

In some embodiments, the disclosure relates to an implementation of a time-domain solution for the wavelet transform of a signal. When implementing the solution, the width of the wavelet function used for the transform (which may have infinite support) may need to be defined. In some embodiments, the wavelet function may be truncated at the defined width. This truncation may cause the real part of the truncated wavelet function to have a non-zero mean. The non-zero mean of the real part of the truncated wavelet function may be forced to have a zero mean. Using the truncated, mean-adjusted wavelet, a CWT is applied to the signal of interest for further analysis.

In some embodiments, the time-based CWT may be applied to a physiological signal. To create the waveform to be used for the CWT, a wavelet may be truncated. The mean of the real part of the truncated wavelet may be adjusted such that it is substantially removed. A continuous wavelet processing system may apply the truncated, mean-adjusted wavelet to the physiological signal using a continuous wavelet transform. Processing hardware and/or software may analyze the transformed signal for physiological information and characteristics, for example, where the physiological signal is a PPG signal, pulse rate, oxygen saturation or any combination thereof may be determined in accordance with the present disclosure.

In some embodiments, the system of the present disclosure may include a receiver for receiving the signal, and processing hardware and software for processing the signal. For example, the processing hardware may include an electronic processor. The system may include memory for storing the signal and other necessary data. The processing hardware and software may be configured to generate a truncated, mean-adjusted wavelet. The processing hardware and software may be configured to transform the signal, or a representation thereof, using a continuous wavelet transform to generate a transformed signal. In some embodiments, the continuous wavelet transform may be applied to the received signal using at least in part the truncated, mean-adjusted wavelet. The processing hardware and software may derive information about the signal from the transformed signal. In some embodiments, the received signal may be a physiological signal.

In some embodiments of the present disclosure, a wavelet, which may have infinite support, may be selected for the time-based continuous wavelet transform. The selected wavelet may be truncated to fit a finite support. In some embodiment, processing hardware and/or software may set the edge values of the wavelet to zero. In some embodiments, processing hardware and/or software may remove the edge values of the wavelet. Using complex analysis, processing hardware and/or software may process the truncated wavelet to find the real part of the truncated wavelet. In some embodiments, the wavelet selected may be a complex wavelet that includes separate real and imaginary parts. In some embodiments, processing hardware and/or software may determine the mean value of the real part of the truncated wavelet and may force the real part of the complex wavelet to have a zero mean. In some embodiments, processing hardware and/or software may remove the non-zero mean from the real part of the wavelet to generate the truncated, mean-adjusted wavelet.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows an illustrative pulse oximetry system in accordance with an embodiment;

FIG. 2 is a block diagram of the illustrative pulse oximetry system of FIG. 1 coupled to a patient in accordance with an embodiment;

FIGS. 3(a) and 3(b) show illustrative views of a scalogram derived from a PPG signal in accordance with an embodiment;

FIG. 3(c) shows an illustrative scalogram derived from a signal containing two pertinent components in accordance with an embodiment;

FIG. 3(d) shows an illustrative schematic of signals associated with a ridge in FIG. 3(c) and illustrative schematics of a further wavelet decomposition of these newly derived signals in accordance with an embodiment;

FIGS. 3(e) and 3(f) are flow charts of illustrative steps involved in performing an inverse continuous wavelet transform in accordance with embodiments;

FIG. 4 is a block diagram of an illustrative continuous wavelet processing system in accordance with some embodiments;

FIG. 5 is a flow chart of illustrative steps involved in performing a continuous wavelet transform based at least in part on a truncated, mean-adjusted wavelet in accordance with some embodiments;

FIG. 6 is a flow chart of illustrative steps involved in generating a truncated, mean-adjusted wavelet in accordance with some embodiments;

FIG. 7 is a flow chart of illustrative steps involved in performing a continuous wavelet transform on a physiological signal in accordance with some embodiments;

FIG. 8(a) shows an illustrative truncated wavelet function having a non-zero mean for the real part in accordance with some embodiments;

FIG. 8(b) shows an illustrative truncated wavelet function with the non-zero mean for the real part removed from the wavelet function shown in FIG. 8(a) in accordance with some embodiments;

FIG. 9 shows an illustrative scalogram derived from a continuous wavelet transform of a physiological signal in accordance with some embodiments;

FIGS. 10(a) and 10(b) show illustrative scalograms derived from time-based continuous wavelet transforms of the same physiological signal used for FIG. 9, using at least in part a Morlet wavelet with a cut-off at three and two standard deviations, respectively, of the Gaussian envelope in accordance with some embodiments;

FIGS. 11(a) and 11(b) show illustrative scalograms derived from the real part and imaginary part, respectively, of the time-based continuous wavelet transforms of the same physiological signal used for FIG. 9, using at least in part a Morlet wavelet with a cut-off at two standard deviations of the Gaussian envelope in accordance with some embodiments; and

FIG. 12 shows an illustrative scalogram of a time-based continuous wavelet transform of the same physiological signal used for FIG. 9, using at least in part a truncated, mean-adjusted wavelet in accordance with some embodiments.

### DETAILED DESCRIPTION

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beefs law. The following notation will be used herein:

I(λ,t)=I_{O}(λ)exp(−(sβ_{O}(λ)+(1−s)βᵣ(λ))l(t)) (1)

where:
λ=wavelength;
t=time;
I=intensity of light detected;
I_{O}=intensity of light transmitted;
s=oxygen saturation;
β_{O}, βᵣ=empirically derived absorption coefficients; and
l(t)=a combination of concentration and path length from emitter to detector as a function of time.

The traditional approach measures light absorption at two wavelengths (e.g., red and infrared (IR)), and then calculates saturation by solving for the “ratio of ratios” as follows.

1. First, the natural logarithm of (1) is taken (“log” will be used to represent the natural logarithm) for IR and Red

log I=log I_{O}−(sβ_{O}+(1−s)βᵣ)l (2)

2. (2) is then differentiated with respect to time

$$\frac{d log I}{d t} = - ( s {β}_{O} + ( 1 - s ) {β}_{r} ) \frac{d l}{d t} \qquad ( 3 )$$

3. Red (3) is divided by IR (3)

$$\frac{d log I ( {λ}_{R} ) / d t}{d log I ( {λ}_{IR} ) / d t} = \frac{s {β}_{O} ( {λ}_{R} ) + ( 1 - s ) {β}_{r} ( {λ}_{R} )}{s {β}_{O} ( {λ}_{IR} ) + ( 1 - s ) {β}_{r} ( {λ}_{IR} )} \qquad ( 4 )$$

### 4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\frac{d log I ( {λ}_{R} )}{d t} ( {β}_{O} ( {λ}_{IR} ) - {β}_{r} ( {λ}_{IR} ) ) - \frac{d log I ( {λ}_{IR} )}{d t} ( {β}_{O} ( {λ}_{R} ) - {β}_{r} ( {λ}_{R} ) )}$$

Note in discrete time

$$\frac{d log I ( λ , t )}{d t} ≃ log I ( λ , {t}_{2} ) - log I ( λ , {t}_{1} )$$

### Using log A-log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{O} ( {λ}_{IR} ) - {β}_{r} ( {λ}_{IR} ) ) - {β}_{O} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{d I / d t}{I} \qquad ( 6 )$$

now (5) becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} \qquad ( 7 )$$

which defines a cluster of points whose slope of y versus x will give R where

x(t)=[I(t₂,λ_{IR})−I(t₁,λ_{IR})]I(t₁,λ_{R})

y(t)=[I(t₂,λ_{R})−I(t₁,λ_{R})]I(t₁,λ_{IR})

y(t)=Rx(t) (8)

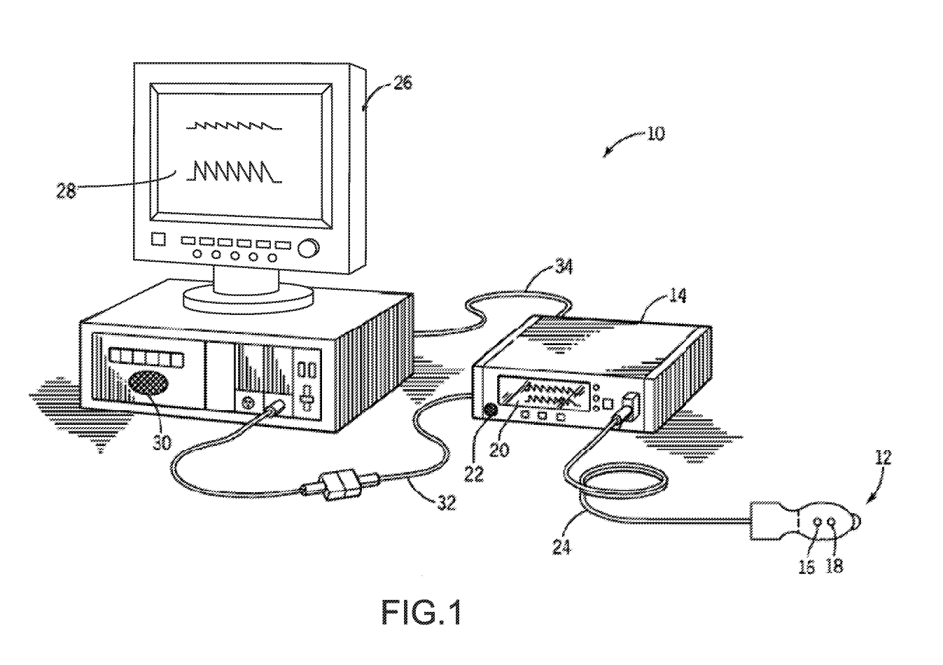

FIG. 1 is a perspective view of an embodiment of a pulse oximetry system 10. System 10 may include a sensor 12 and a pulse oximetry monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

According to another embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patient's forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the oximetry reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multiparameter patient monitor 26 may be configured to display an estimate of a patient's blood oxygen saturation generated by pulse oximetry monitor 14 (referred to as an “SpO₂” measurement), pulse rate information from monitor 14 and blood pressure from a blood pressure monitor (not shown) on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

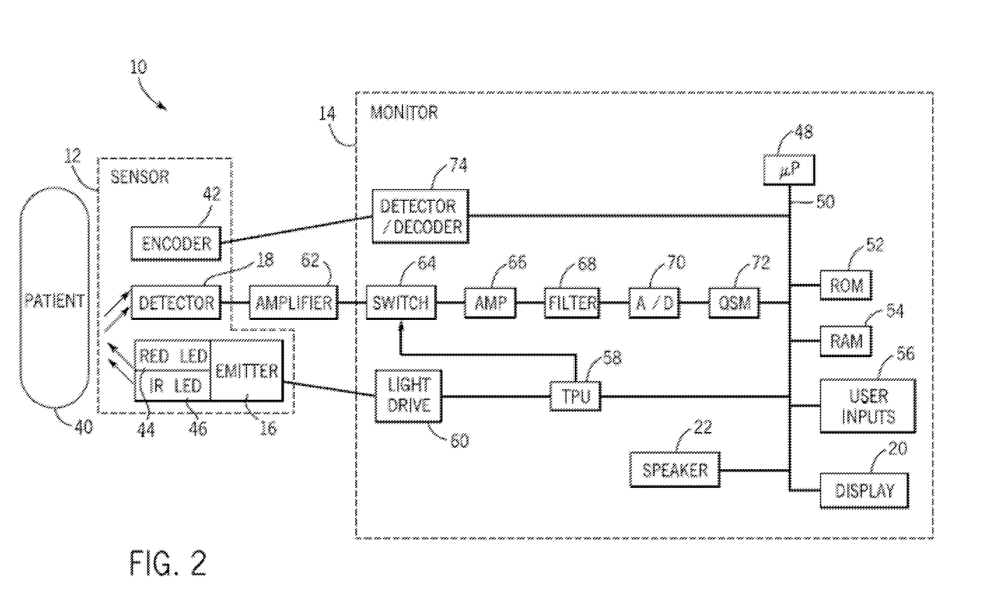

FIG. 2 is a block diagram of a pulse oximetry system, such as pulse oximetry system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit at least two wavelengths of light (e.g., RED and IR) into a patient's tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In one embodiment, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the RED and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the RED and IR wavelengths in the patient's tissue 40.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as SpO₂ and pulse rate, using various algorithms and/or look-up tables based on the value of the received signals and/or data corresponding to the light received by detector 18. Signals corresponding to information about patient 40, and particularly about the intensity of light emanating from a patients tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a pulse oximetry signal relied upon by a physician, without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

It will be understood that the present disclosure is applicable to any suitable signals and that PPG signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In one embodiment, a PPG signal may be transformed using a continuous wavelet transform. Information derived from the transform of the PPG signal (i.e., in wavelet space) may be used to provide measurements of one or more physiological parameters.

The continuous wavelet transform of a signal x(t) in accordance with the present disclosure may be defined as

$$T ( a , b ) = \frac{1}{\sqrt{a}} {\int }_{- ∞}^{+ ∞} x ( t ) {ψ}^{*} ( \frac{t - b}{a} ) d t \qquad ( 9 )$$

where ψ*(t) is the complex conjugate of the wavelet function ψ(t), a is the dilation parameter of the wavelet and b is the location parameter of the wavelet. The transform given by equation (9) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The continuous wavelet transform decomposes a signal using wavelets, which are generally highly localized in time. The continuous wavelet transform may provide a higher resolution relative to discrete transforms, thus providing the ability to garner more information from signals than typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example, any type of Fourier transform would generate such a two-dimensional spectrum. In contrast, wavelet transforms, such as continuous wavelet transforms, are required to be defined in a three-dimensional coordinate system and generate a surface with dimensions of time, scale and, for example, amplitude. Hence, operations performed in a spectral domain cannot be performed in the wavelet domain; instead the wavelet surface must be transformed into a spectrum (i.e., by performing an inverse wavelet transform to convert the wavelet surface into the time domain and then performing a spectral transform from the time domain). Conversely, operations performed in the wavelet domain cannot be performed in the spectral domain; instead a spectrum must first be transformed into a wavelet surface (i.e., by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g., to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (often in time) and scale.

The energy density function of the wavelet transform, the scalogram, is defined as

S(a,b)=|T(a,b)|² (10)

where ‘∥’is the modulus operator. The scalogram may be resealed for useful purposes. One common resealing is defined as

$${S}_{R} ( a , b ) = \frac{{\vert T ( a , b ) \vert}^{2}}{a} \qquad ( 11 )$$

and is useful for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane are labeled a “maxima ridge”.

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and then multiplying the result by \sqrt{a}.

In the discussion of the technology which follows herein, the “scalogram” may be taken to include all suitable forms of resealing including, but not limited to, the original unsealed wavelet representation, linear resealing, any power of the modulus of the wavelet transform, or any other suitable resealing. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram”.

A scale, which may be interpreted as a representative temporal period, may be converted to a characteristic frequency of the wavelet function. The characteristic frequency associated with a wavelet of arbitrary a scale is given by

$$f = \frac{{f}_{c}}{a} \qquad ( 12 )$$

where f_{c}, the characteristic frequency of the mother wavelet (i.e., at a=1), becomes a scaling constant and f is the representative or characteristic frequency for the wavelet at arbitrary scale a.

Any suitable wavelet function may be used in connection with the present disclosure. One of the most commonly used complex wavelets, the Morlet wavelet, is defined as:

ψ(t)=π^{-1/4}(e^{i2πf}^{₀}^{t}−e^{-(2πf}^{₀}⁾^{²}^{/2})e^{-t}^{²}^{/2} (13)

where f₀ is the central frequency of the mother wavelet. The second term in the parenthesis is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as

$$ψ ( t ) = \frac{1}{{π}^{1 / 4}} {e}^{i2π {f}_{0} t} {e}^{- {t}^{2} / 2} \qquad ( 14 )$$

This wavelet is a complex wave within a scaled Gaussian envelope. While both definitions of the Morlet wavelet are included herein, the function of equation (14) is not strictly a wavelet as it has a non-zero mean (i.e., the zero frequency term of its corresponding energy spectrum is non-zero). However, it will be recognized by those skilled in the art that equation (14) may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be extracted from the wavelet decomposition of signals. For example, wavelet decomposition of PPG signals may be used to provide clinically useful information within a medical device.

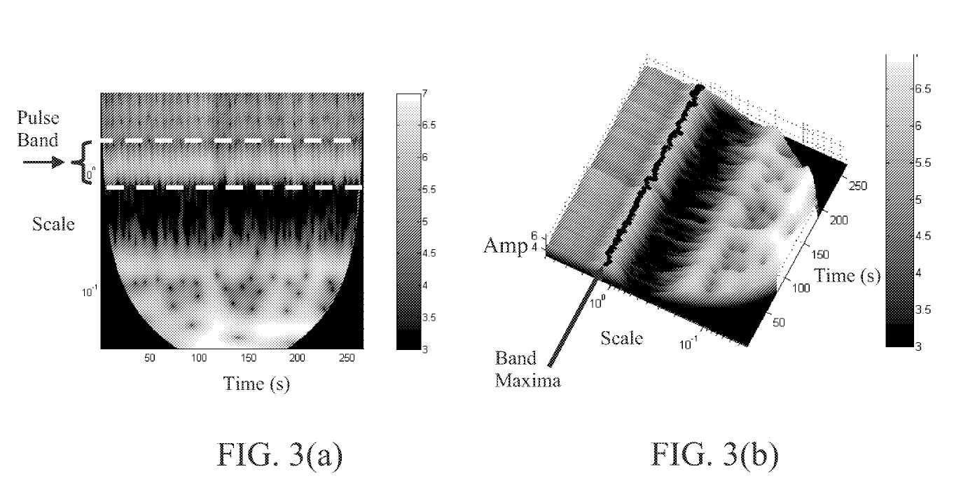

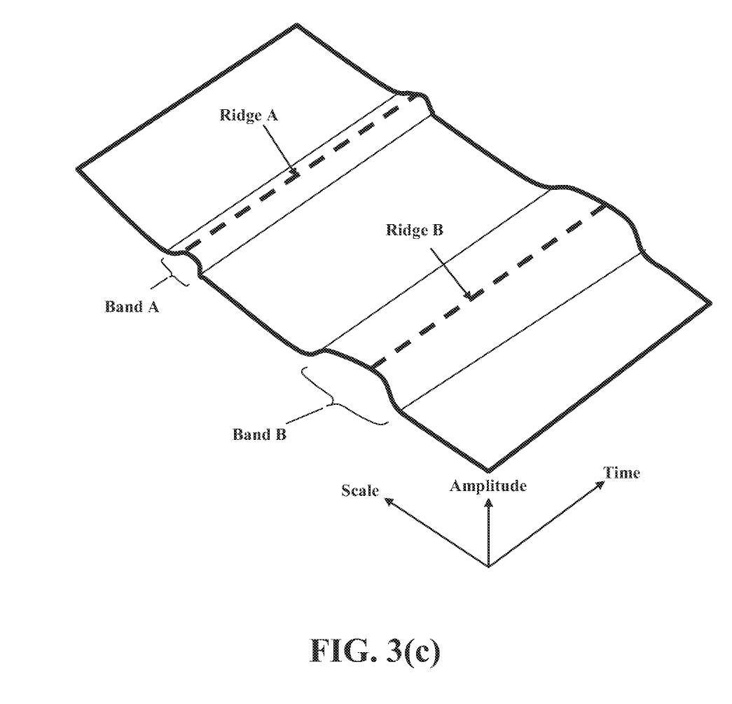

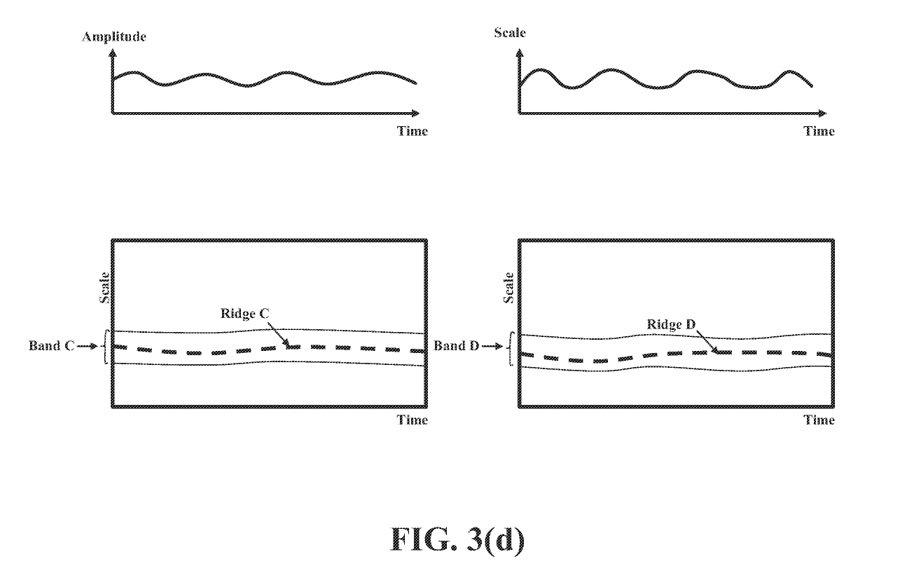

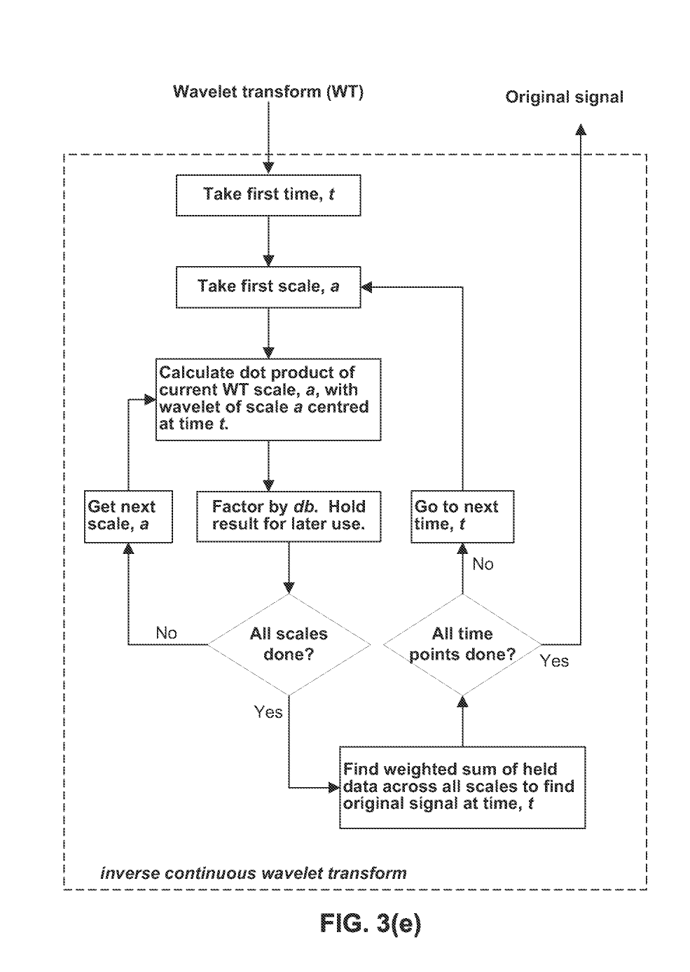

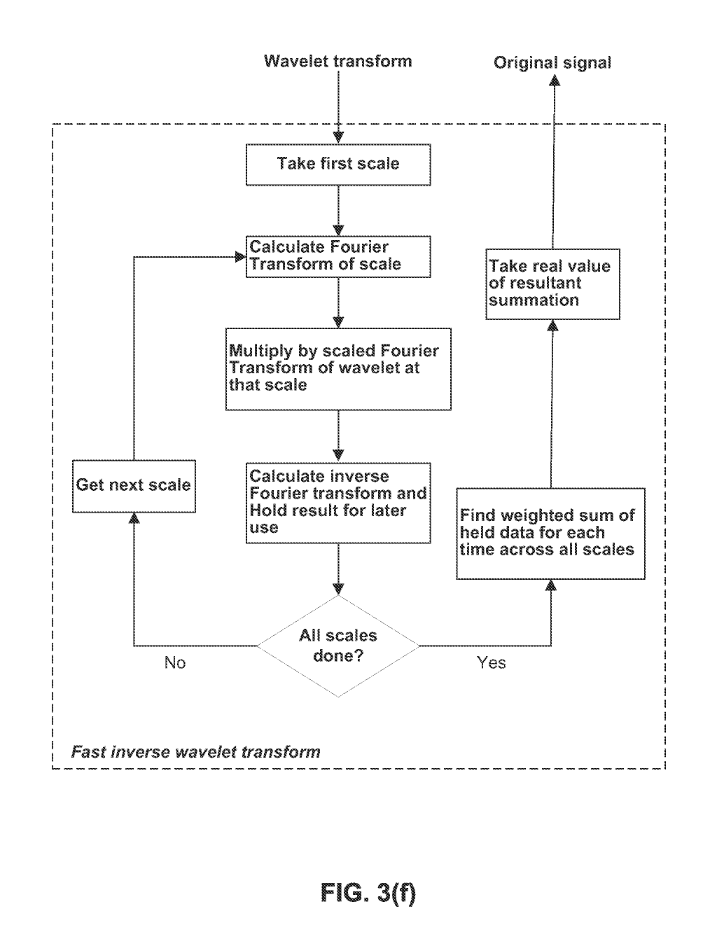

Pertinent repeating features in a signal give rise to a time-scale band in wavelet space or a resealed wavelet space. For example, the pulse component of a PPG signal produces a dominant band in wavelet space at or around the pulse frequency. FIGS. 3(a) and (b) show two views of an illustrative scalogram derived from a PPG signal, according to an embodiment. The figures show an example of the band caused by the pulse component in such a signal. The pulse band is located between the dashed lines in the plot of FIG. 3(a). The band is formed from a series of dominant coalescing features across the scalogram. This can be clearly seen as a raised band across the transform surface in FIG. 3(b) located within the region of scales indicated by the arrow in the plot (corresponding to 60 beats per minute). The maxima of this band with respect to scale is the ridge. The locus of the ridge is shown as a black curve on top of the band in FIG. 3(b). By employing a suitable resealing of the scalogram, such as that given in equation (11), the ridges found in wavelet space may be related to the instantaneous frequency of the signal. In this way, the pulse rate may be obtained from the PPG signal. Instead of resealing the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. In this way, both times between individual pulses and the timing of components within each pulse may be monitored and used to detect heart beat anomalies, measure arterial system compliance, or perform any other suitable calculations or diagnostics. Alternative definitions of a ridge may be employed. Alternative relationships between the ridge and the pulse frequency of occurrence may be employed.

As discussed above, pertinent repeating features in the signal give rise to a time-scale band in wavelet space or a resealed wavelet space. For a periodic signal, this band remains at a constant scale in the time-scale plane. For many real signals, especially biological signals, the band may be non-stationary; varying in scale, amplitude, or both over time. FIG. 3(c) shows an illustrative schematic of a wavelet transform of a signal containing two pertinent components leading to two bands in the transform space, according to an embodiment. These bands are labeled band A and band B on the three-dimensional schematic of the wavelet surface. In this embodiment, the band ridge is defined as the locus of the peak values of these bands with respect to scale. For purposes of discussion, it may be assumed that band B contains the signal information of interest. This will be referred to as the “primary band”. In addition, it may be assumed that the system from which the signal originates, and from which the transform is subsequently derived, exhibits some form of coupling between the signal components in band A and band B. When noise or other erroneous features are present in the signal with similar spectral characteristics of the features of band B then the information within band B can become ambiguous (i.e., obscured, fragmented or missing). In this case, the ridge of band A may be followed in wavelet space and extracted either as an amplitude signal or a scale signal which will be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively. The top plots of FIG. 3(d) show a schematic of the RAP and RSP signals associated with ridge A in FIG. 3(e). Below these RAP and RSP signals are schematics of a further wavelet decomposition of these newly derived signals. This secondary wavelet decomposition allows for information in the region of band B in FIG. 3(c) to be made available as band C and band D. The ridges of bands C and D may serve as instantaneous time-scale characteristic measures of the signal components causing bands C and D. This technique, which will be referred to herein as secondary wavelet feature decoupling (SWFD), may allow information concerning the nature of the signal components associated with the underlying physical process causing the primary band B (FIG. 3(c)) to be extracted when band B itself is obscured in the presence of noise or other erroneous signal features.

In some instances, an inverse continuous wavelet transform may be desired, such as when modifications to a scalogram (or modifications to the coefficients of a transformed signal) have been made in order to, for example, remove artifacts. In one embodiment, there is an inverse continuous wavelet transform which allows the original signal to be recovered from its wavelet transform by integrating over all scales and locations, a and b:

$$x ( t ) = \frac{1}{{C}_{g}} {\int }_{- ∞}^{∞} {\int }_{0}^{∞} T ( a , b ) \frac{1}{\sqrt{a}} ψ ( \frac{t - b}{a} ) \frac{d a d b}{{a}^{2}} \qquad ( 15 )$$

which may also be written as:

$$x ( t ) = \frac{1}{{C}_{g}} {\int }_{- ∞}^{∞} {\int }_{0}^{∞} T ( a , b ) {ψ}_{a , b} ( t ) \frac{d a d b}{{a}^{2}} \qquad ( 16 )$$

where C_{g} is a scalar value known as the admissibility constant. It is wavelet type dependent and may be calculated from:

$${C}_{g} = {\int }_{0}^{∞} \frac{{\vert \hat{ψ} ( f ) \vert}^{2}}{f} d f \qquad ( 17 )$$

FIG. 3(e) is a flow chart of illustrative steps that may be taken to perform an inverse continuous wavelet transform in accordance with the above discussion. An approximation to the inverse transform may be made by considering equation (15) to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 3(f) is a flow chart of illustrative steps that may be taken to perform an approximation of an inverse continuous wavelet transform. It will be understood that any other suitable technique for performing an inverse continuous wavelet transform may be used in accordance with the present disclosure.

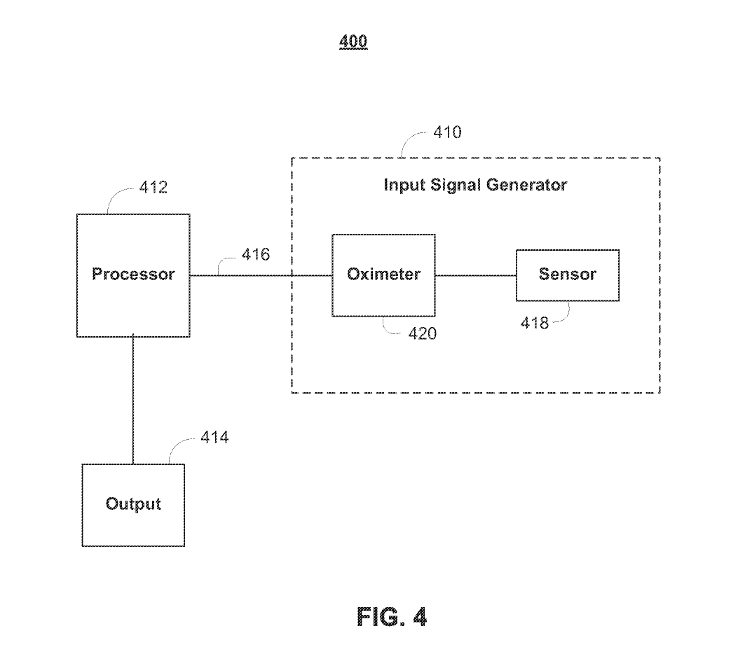

FIG. 4 is an illustrative continuous wavelet processing system in accordance with an embodiment. In this embodiment, input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data, signal generating equipment, or any combination thereof to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In this embodiment, signal 416 may be coupled to processor 412. Processor 412 may be any suitable software, firmware, and/or hardware, and/or combinations thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the continuous wavelet transforms of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, and/or any other suitable filtering, and/or any combination thereof.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to a continuous wavelet transform of input signal 416, such as data representing a scalogram. In one embodiment, data representing a scalogram may be stored in RAM or memory internal to processor 412 as any suitable three-dimensional data structure such as a three-dimensional array that represents the scalogram as energy levels in a time-scale plane. Any other suitable data structure may be used to store data representing a scalogram.

Processor 412 may be coupled to output 414. Output 414 may be any suitable output device such as, for example, one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 may be implemented as parts of sensor 12 and monitor 14 and processor 412 may be implemented as part of monitor 14.

To perform a time-domain continuous wavelet transform, the signal of interest may be convolved with a wavelet. When implementing a time-domain solution for the continuous wavelet transform of a signal, the width of the wavelet function may be truncated at a defined cut-off length to fit a finite support. In some embodiments, the original wavelet function may have theoretically infinite support, and the truncation process may cause the real part of the truncated wavelet to have a non-zero mean.

In some embodiments, the signal of interest, for example, a physiological signal, may have a significant non-zero mean. When the signal of interest is convolved with a wavelet having even a relatively small mean, a significant artifact may manifest in the resulting convolution, caused at least in part by the non-zero mean of the wavelet. To reduce the effects of the truncation caused by the convolution, the non-zero mean of the truncated may be artificially adjusted and/or removed prior to convolution.

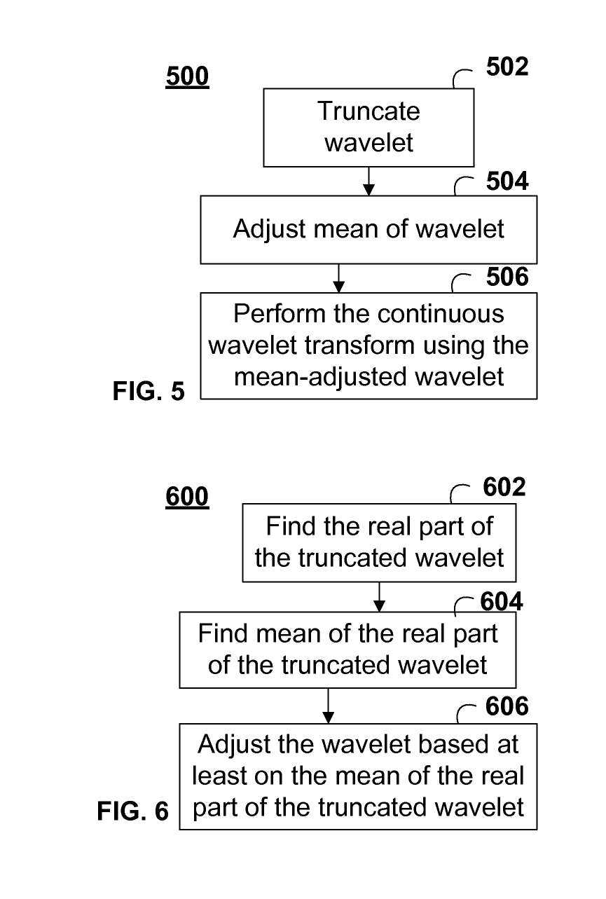

FIG. 5 is a flow chart of illustrative steps involved in performing a time-based continuous wavelet transform based at least in part on a truncated, mean-adjusted wavelet. A wavelet may be truncated (step 502 of FIG. 5). In order to reduce at least some of the artifacts introduced by the truncation, the real part of the complex wavelet may be forced to have zero mean (step 504 of FIG. 5) to generate a truncated, mean-adjusted wavelet. Using at least in part the truncated, mean-adjusted wavelet, a continuous wavelet transform may be applied to transform the signal (step 506 of FIG. 5).

In some embodiments, the continuous wavelet transform may be based at least in part on a Morlet wavelet. The Morlet wavelet, which may include a sine wave localized by a Gaussian window, may be truncated at, for example, three standard deviations on either side of the wavelet center. The truncation step may be performed by removing edge values beyond the defined cut-off or by setting the edge values beyond the cut-off to zero or by modifying the wavelet function in such a way that the function goes to zero at, and beyond, the truncation edge. In general, at three standard deviations, a Morlet wavelet may have near zero values prior to truncation, and truncation may preserve most of the energy of the wavelet. It will be understood that any suitable cut-offs may be used, such as three standard deviations, three and a half standard deviations, any other suitable number of standard deviations, or any combination thereof. It will be understood that any other suitable wavelets may be used, such as Modified Morlet wavelet. Mexican hat wavelet, Complex Mexican hat wavelet, Shannon wavelet, Difference of Gaussians, Hermitian wavelet, Hermitian hat wavelet, Beta wavelet, Causal Wavelet, μ wavelets, Cauchy wavelet, Addison wavelet, and/or any combination or variation thereof.

In some embodiments, truncation may be performed by multi-parameter patient monitor 26 of pulse oximetry system 10 of FIG. 1, monitor 14 of FIG. 2, or processor 412 of FIG. 4. In some embodiments, the signal may be a physiological signal such as a PPG signal, which may be generated using, for example, components described in relation to pulse oximetry system 10 and input signal generator 410. The transformed PPG signal may be processed to extract physiological information and/or characteristics, as discussed in relation to, for example, FIGS. 3(a), 3(b), 3(c) and 3(d).

In some embodiments, the truncation process, such as indicated at step 502 of FIG. 5, may cause significant numerical errors when the truncated wavelet is convolved with the signal of interest to generate a wavelet transform. The significant numerical errors may be caused by the non-zero mean that manifests in the real part of the truncated wavelet function. Though small, the non-zero mean may lead to large errors when convolved with signals and/or signal segments, for example, a physiological signal, which have significant non-zero means.

To reduce the effects of truncation, the wavelet may be examined using complex analysis to separate the real and imaginary parts of the wavelet. The real part of the wavelet may be analyzed to determine the mean. For example, integration on the values of the real part of the complex wavelet divided by the length of its domain may be used at least in part for determining the mean. It will be understood that any suitable technique for determining the mean may be used. After determining the mean, the mean value of the real part of the wavelet may be subtracted from the real part of the complex wavelet to force the real part to have zero mean (e.g., step 504 of FIG. 5).

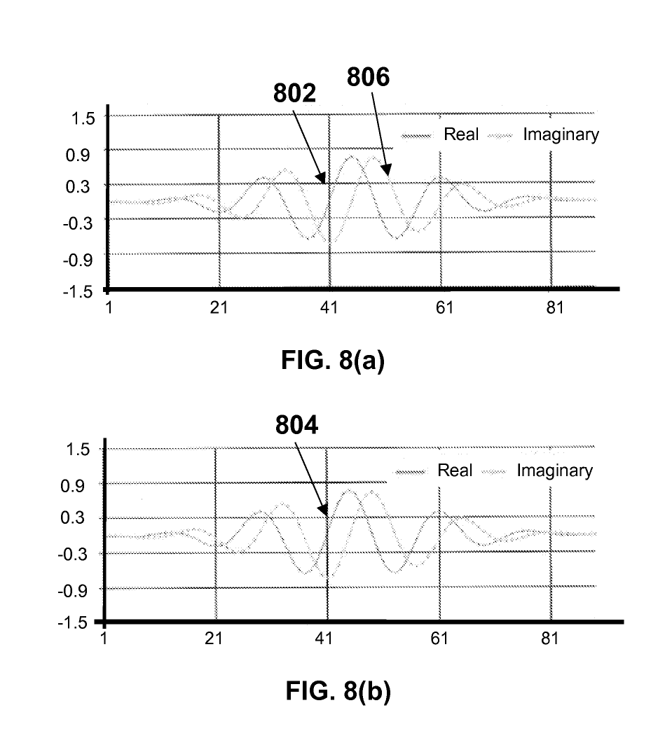

In some embodiments, the mean value of the real part of the truncated wavelet is forced to be zero by subtracting the mean value from the real part of the truncated wavelet. It will be understood that any suitable methods for adjusting mean may be used, such as any suitable calculation that modifies the function so that it becomes zero at and beyond the defined truncated edge of the wavelet while setting the mean value to zero. The effects of wavelet truncation in accordance with the present disclosure are illustrated in, for example, FIGS. 8(a) and 8(b) which are described in more detail below.

FIG. 6 is a flow chart of illustrative steps involved in generating a truncated, mean-adjusted wavelet in accordance with some embodiments of the present disclosure. The truncated wavelet may be separated into its real and imaginary parts (step 602 of FIG. 6). In some embodiments, the real part of the truncated wavelet may be determined based at least in part on complex analysis. Processing hardware, software, or both such as multi-parameter patient monitor 26 of pulse oximetry system 10 of FIG. 1, monitor 14 of FIG. 2, processor 412 of FIG. 4, or any combination thereof may calculate the mean of the real part of the truncated wavelet (step 604 of FIG. 6) using any suitable method for calculating the mean value of the real part of the truncated wavelet. For example, integrating the real part of the truncated wavelet over the domain divided by the length of the domain may yield the average/mean value of the real part of the truncated wavelet. Any suitable methods, or combination thereof, may be used, including, for example, a weighted average.

Using at least in part the mean value, processing hardware, software, or both such as multi-parameter patient monitor 26 of pulse oximetry system 10 of FIG. 1, monitor 14 of FIG. 2, processor 412 of FIG. 4, or any combination thereof may adjust the mean of the wavelet (step 606 of FIG. 6) to reduce the effects caused by the truncation. The real part of the truncated, complex wavelet may be forced to have a zero mean, using any suitable mean adjustment process such as step 504 of FIG. 5. For instance, the mean value may be subtracted from the real part of the truncated, complex wavelet. Employing illustrative process 600, a truncated, mean adjusted wavelet (e.g., wavelet 804 of FIG. 8(b)), may be generated from the truncated wavelet (e.g., wavelet 802 of FIG. 8(a). Note that FIGS. 8(a) and 8(b) may look identical to the eye. This is because the difference in the mean values of the real part is very small compared to the amplitude of the wavelet function. However, even this small difference may cause large errors when the wavelet is convolved with a signal with a significant non-zero mean.

Processes 500 and 600, or any substep or combination therein, may be performed on physiological signals using multi-parameter patient monitor 26 of pulse oximetry system 10 of FIG. 1, monitor 14 of FIG. 2 or processor 412 of FIG. 4.

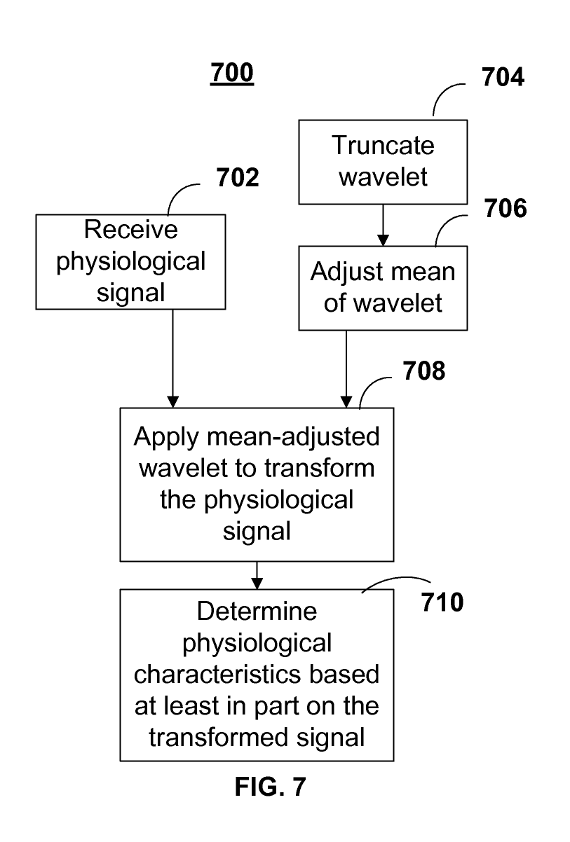

FIG. 7 is a flow chart of illustrative steps involved in performing a continuous wavelet transform on a physiological signal in accordance with some embodiments of the present disclosure. A physiological signal, such as a PPG signal, may be received (step 702 of FIG. 7), for example, in a manner described with regard to FIGS. 1 and 2. Processing hardware and/or software may truncate a wavelet (step 704 of FIG. 7), for example, in a manner described above with regard to FIGS. 5 and 6. Processing hardware and/or software may adjust the mean of the wavelet (step 706 of FIG. 7). The physiological signal may be transformed using at least in part the truncated, mean-adjusted wavelet (step 708 of FIG. 7), for example, by employing a time-based CWT method. The physiological information/characteristics may be extracted/determined based at least in part on the transformed signal (step 710 of FIG. 7), in manners discussed above with respect to, for example, FIGS. 3(a), 3(b), 3(c) and 3(d).

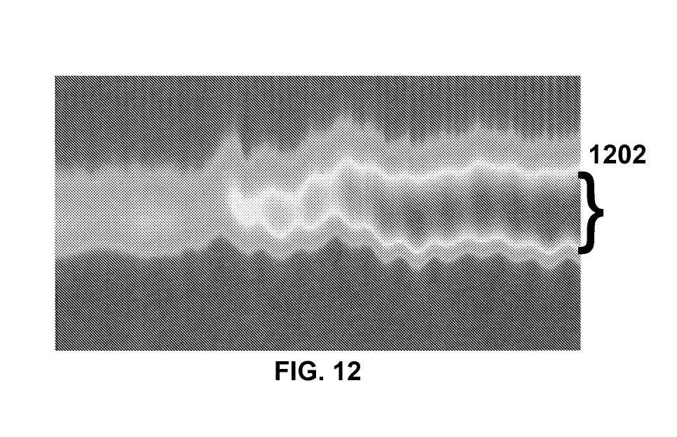

One or more of the steps shown in process 700 of FIG. 7 may be performed by pulse oximetry system 10 of FIGS. 1 and 2, or system 400 of FIG. 4. Illustrative results of applying process 700 (e.g., performing a continuous wavelet transform on a PPG signal using a truncated, mean-adjusted wavelet) is shown in a scalogram in FIG. 12, where a high energy pulse band may be seen in region 1202.

In some embodiments in accordance with the present disclosure, a complex wavelet may be used, such as complex Morlet wavelet. To illustrate the differences between the truncated wavelet and the truncated, mean-adjusted wavelet, two separate complex wavelets are shown in FIGS. 8(a) and 8(b). In FIG. 8(a), an illustrative truncated complex Morlet wavelet function having a non-zero mean for the real part 802 is shown. In comparison, FIG. 8(b) shows an illustrative, mean-adjusted, truncated wavelet function with the non-zero mean for the real part 804 removed from the real part 802 of the wavelet function shown in FIG. 8(a). Illustrative ways to force the non-zero mean wavelet to have a zero mean are described in relation to FIGS. 5, 6 and 7. Any other suitable processes may be employed to truncate and/or remove the mean of the wavelet function.

In some embodiments, the adjustment on the mean may be small. The example complex wavelet shown in FIG. 8(a) (with a central characteristic frequency of 5.5 truncated at a width of three standard deviations) has a real part 802 that has mean value of −0.000233875 (−0.03% of the maximum value), illustrating that a tiny mean error may cause significant numerical problem in the analysis. The mean value of the real part 802 may be significant, however, especially when compared to the negligibly small mean value of the imaginary part 806 (5.19929760917768×10⁻¹⁷).

Performing a transform using a truncated, mean-adjusted wavelet may change the overall energy contained in the wavelet. However, the energy changes are likely to be relatively small in practice (the difference in computed energy for the wavelets of FIGS. 8(a) and 8(b) is down to the 7th significant figure). Even with a narrow wavelet width of one standard deviation, energy changes introduced by mean-subtraction is calculated at below 1%.

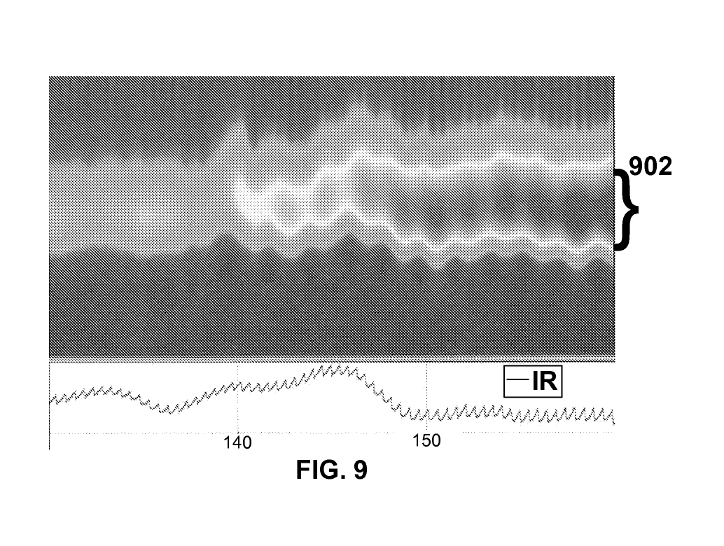

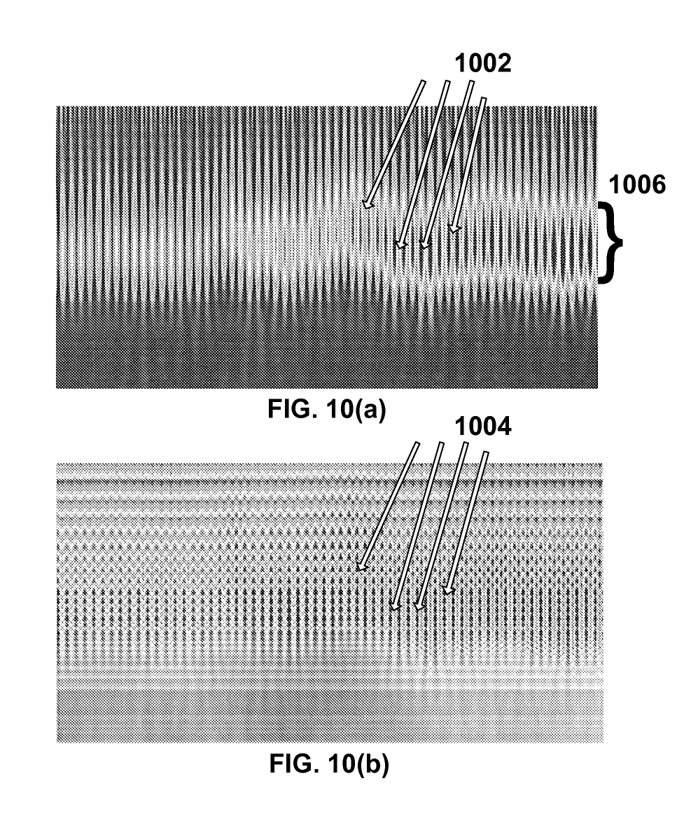

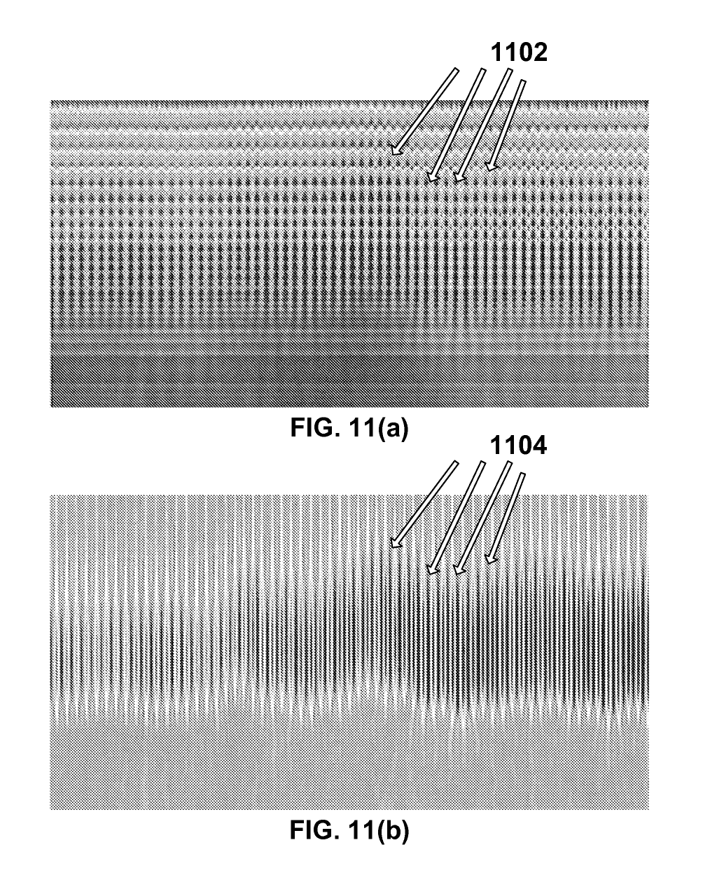

To illustrate the effects of truncation, a baseline continuous wavelet transform of a PPG signal is shown in the scalogram of FIG. 9. For purposes of illustration, the physiological signal used is an IR PPG captured using a pulse oximeter device at 75 Hz, A high energy pulse band may can be seen across the middle of the transform plot in region 902. FIGS. 10(a) and 10(b) show illustrative scalograms derived from continuous wavelet transforms of the same PPG signal used for FIG. 9 using a Morlet wavelet with a cut-off at three and two standard deviations, respectively, of the Gaussian envelope. When compared with FIG. 9, it may be noticeable from the plot that the pulse band across the signal may have broken up, taking up a cycling appearance of a real (or imaginary) part of the transform rather than the modulus. When more of the edges of the wavelet is cut off (i.e., cut-off is set at two standard deviations rather than at three standard deviations), the resulting scalogram as seen in FIG. 10(b) is erroneous. Upon closer inspection with complex analysis of the result of the transform shown in FIG. 10(b), the imaginary part appears correct with regular phase cycling across the band (see features 1104 of FIG. 11(b)), and the real part appears erroneous (see artifacts 1102 of FIG. 11(a)) where a pulse band can no longer be seen.

In some embodiments, the erroneous effects of the truncation on the transform may be mitigated by increasing the wavelet width (e.g., increasing the cut-off value). However, using a wider wavelet may require more processing power and may increase the number of samples required to fully resolve the continuous wavelet transform. Conversely, by decreasing the wavelet width, the effects of truncation may be amplified, as illustrated by a visual comparison of scalograms shown in FIGS. 10(a) and 10(b). At three standard deviations, artifacts 1002 may begin to break up the high energy pulse band in region 1006. At two standard deviations, the artifacts 1004 caused by the truncation process may worsen and lead to an erroneous plot.

The effects of truncation, as discussed with regard to FIGS. 10(a), 10(b), 11(a) and 11(b), may be caused by the truncation of the wavelet to fit a finite support. For example, truncation may affect the transform when the cut-off was equal to three or two standard deviations of the Gaussian envelope containing the Morlet wavelet function. To show the dominant effect truncation may have on the real part of the transformed signal, FIGS. 11(a) and 11(b) show illustrative scalograms derived from the real part and imaginary part, respectively, of the time-based continuous wavelet transform seen in the scalogram of FIG. 10(b) (of the same physiological signal used in the baseline example of FIG. 9). The transform shown in 11(a) and 11(b) was generated using at least in part a Morlet wavelet with a cut-off at two standard deviations of the Gaussian envelope. The truncation may cause the real part of the wavelet, for example, as seen in wave 802 of FIG. 8(a), to have a slight non-zero mean, which when convolved with a signal with large baseline values causes large errors in the resulting transform (see artifacts 1102 of FIG. 11(a)). The imaginary part of the resulting transform, for example, as illustrated in FIG. 11(b), may not suffer from this effect because the imaginary part (e.g., wave 806 of FIG. 8(a)) may be rotationally symmetric about the origin of the wavelet so all truncation values are equal and opposite about zero (i.e., the imaginary part have an insignificant non-zero mean). As confirmed by the scalogram seen in FIG. 11(b), artifacts 1104 seen in FIG. 11(b) are relatively minor as compared to artifacts 1102.

Note that the above description may also be applied to truncated wavelets that have imaginary parts with non-zero means, or wavelets that have imaginary and real parts with non-zero means. Those skilled in the art will appreciate that the process described above may be applied advantageously to the real, imaginary or both parts of a wavelet that has been truncated in time (or altered in another way for example truncated in amplitude) to facilitate its practical implementation.

FIG. 12 shows an illustrative scalogram of a continuous wavelet transform of the same physiological signal used for FIG. 9, using at least in part a truncated, mean-adjusted wavelet (cut-off at three standard deviations). By comparing the scalogram shown in FIG. 12 and the baseline scalogram shown in FIG. 9, the effects of truncation has been substantially resolved. In this illustration, a high energy pulse band is visually apparent in region 1202.

The techniques described herein may be applied to real-time continuous wavelet transform computation where a real-time convolution is performed. The method may be used to compute the continuous wavelet transform of a PPG signal in order that a physiological parameter may be determined, for example SpO₂, respiration rate, respiration effort, any other suitable physiological parameters or any combination thereof. Those skilled in the art may modify the wavelets to have zero mean using methods other than simply removing the mean value from all points of the wavelet. Techniques that provide a wavelet with zero mean while forcing the truncated edges of the wavelet to have zero values may be used.

The above described embodiments of the present disclosure are presented for purposes of illustration and not of limitation, and the present disclosure is limited only by the claims which follow.

What is claimed is:

1. A method for transforming a signal using a continuous wavelet transform, the method comprising:
generating, using electronic processing equipment, a truncated wavelet from an original wavelet, wherein at least part of the truncated wavelet has a non-zero mean;
forcing, using the electronic processing equipment, the mean of the part of the truncated wavelet to zero to generate a mean-adjusted wavelet; and
performing, using the electronic processing equipment, the continuous wavelet transform on the signal using the mean-adjusted wavelet to generate a transformed signal.

2. The method of claim 1, wherein the part of the truncated wavelet comprises the real part, and/or the imaginary part, and/or a combination thereof.

3. The method of claim 1, wherein generating a truncated wavelet comprises:
forcing, using the electronic processing equipment, the edge values of the wavelet to zero.

4. The method of claim 1, wherein generating a truncated wavelet comprises:
applying, using the electronic processing equipment, a Gaussian envelope to the original wavelet.

5. The method of claim 1, wherein the original wavelet comprises a Morlet wavelet.

6. The method of claim 1, wherein performing the continuous wavelet transform comprises performing a Fast Fourier Transform (FFT)-based method using the electronic processing equipment.

7. The method of claim 1, wherein the signal is a photoplethysmograph signal.

8. The method of claim 1, wherein forcing the mean of the part of the truncated wavelet to zero comprises:
removing the mean value, using the electronic processing equipment, from the part of the truncated wavelet.

9. A system for transforming a signal using a continuous wavelet transform, the system comprising:
an electronic processing equipment configured to:
generate a truncated wavelet from an original wavelet, wherein a part of the truncated wavelet has a non-zero mean;
force the mean of at least part of the truncated wavelet to zero to generate a mean-adjusted wavelet; and
perform the continuous wavelet transform on the signal using the mean-adjusted wavelet to generate a transformed signal.

10. The system of claim 9, wherein the part of the truncated wavelet comprises the real part, and/or the imaginary part, and/or a combination thereof.

11. The system of claim 9, wherein generating a truncated wavelet comprises:
forcing, using the electronic processing equipment, the edge values of the wavelet to zero.

12. The system of claim 9, wherein generating a truncated wavelet comprises:
applying, using the electronic processing equipment, a Gaussian envelope to the original wavelet.

13. The system of claim 9, wherein the original wavelet comprises a Morlet wavelet.

14. The system of claim 9, wherein performing the continuous wavelet transform comprises performing a Fast Fourier Transform (FFT)-based method.

15. The system of claim 9, wherein the signal is a photoplethysmograph signal.

16. The system of claim 9, wherein forcing the mean of the part of the truncated wavelet to zero comprises:
removing the mean value, using the electronic processing equipment, from the part of the truncated wavelet.

17. A method for determining physiological information from a photoplethysmograph (PPG) signal, the method comprising:
collecting the PPG signal using an oximeter;
transforming using electronic processing equipment the PPG signal based at least in part on a continuous wavelet transform wherein the transforming comprises:
truncating a wavelet to generate a truncated wavelet,
forcing the mean of at least part of the truncated wavelet to zero to generate a mean-adjusted wavelet, and
generating a wavelet transform of the PPG signal using the mean-adjusted wavelet; and
determining, using the electronic processing equipment, the physiological information based at least in part on the wavelet transform of the PPG signal.

18. The method of claim 17, wherein the part of the truncated wavelet comprises the real part, and/or the imaginary part, and/or a combination thereof.

19. The method of claim 17, wherein generating a truncated wavelet comprises:
forcing, using the electronic processing equipment, the edge values of the wavelet to zero.

20. The method of claim 17, wherein forcing the mean of the part of the truncated wavelet to zero comprises:
removing the mean value from the part of the truncated wavelet.
